(19) United States
(12) Patent Application Publication
(10) Pub. No.: US 2018/0317834 A1
(43) Pub. Date: Nov. 8, 2018

(54) Maternity Monitoring Device and Method

(71) Applicant: Narmadha Kuppuswami, Wheaton, IL (US)

(72) Inventor: Narmadha Kuppuswami, Wheaton, IL (US)

(21) Appl. No.: 15/585,315

(22) Filed: May 3, 2017

(51) Int. Cl. A61B 5/00; A61B 5/0205

(52) U.S. Cl. CPC A61B 5/4343; A61B 5/742; A61B 5/02055; A61B 5/746; A61B 5/0816; A61B 5/002; A61B 5/6823; A61B 5/6824; A61B 5/0022

(57) ABSTRACT

A monitoring device for monitoring a plurality of parameters of a patient that includes a first sensor placed on the chest wall of a patient, a cord connecting the first sensor to a second sensor placed on a major artery of the upper arm of the patient, and an inflatable cuff placed around the upper arm of the patient. The device includes a screen that displays one of the plurality of parameters and the date and time the parameter was measured. The device can include an automatic messaging system and an automatic alert system, functioning as a standalone unit. When connected to a computer or mobile device, medical personnel can access an obstetric electronic health system to obtain medical guidelines based on alerts from the device. Personnel with the device at a remote center can communicate with a designated center with emergency obstetric care through this electronic health system.

### TECHNICAL FIELD

This disclosure relates to a monitoring device and method, and more particularly, to a maternity monitoring device and method.

### BACKGROUND

Every day approximately 830 women die from preventable causes related to pregnancy and childbirth. About 99% of all maternal deaths occur in developing countries. Maternal mortality is higher in women living in rural areas and among poor communities due to the lack of available resources. However, low resource availability is not the only reason for maternal deaths. Even in the United States, the total number of maternal deaths continues to increase, from 7.2 per 100,000 live births in 1987 to 17.8 per 100,000 live births in 2011. Even more concerning is the fact that for every maternal death, about 100 women will suffer severe maternal morbidity. These women who have gone through severe morbidity may have a similar diagnosis and similar rates of preventable factors as the mothers who did not survive.

Factors that contribute to maternal death in low resources settings most likely are failure to recognize the seriousness of a patient's condition, lack of knowledge, failure to follow recommended practices, lack of or failure to implement policies, protocols and guidelines, and poor organizational arrangements. On the contrary, in developed nations, where skilled obstetrical care is the norm, problems like diagnostic errors, delay in diagnosis, inappropriate or inadequate treatment, and an inability to get the skilled obstetrician at the patient's bedside during acute emergencies are probably contributing to maternal death. Breakdowns in communication and failures of organizational culture and teamwork have consistently ranked among the top three leading contributors to reported maternal and newborn sentinel events.

Women who ultimately die during childbirth rarely go directly from being in a healthy state to death, but rather go through situations that may lead to severe morbidity when necessary aggressive management is not instituted. Typically, there is a transition period where the patient remains seemingly stable, but abnormal vital signs suggest the potential for impending deterioration. This transition period is the crucial time of intervention in order to prevent death. Intervention in this transition period can save the mother's life, but unfortunately this critical transition period is often under-recognized. These vital signs include, but are not limited to, blood pressure, heart rate, respiratory rate, and temperature. A wearable device, which can measure these signs, paired with clinical decision support capabilities can assist care providers, especially those in low resource settings and with less experience, in initiating lifesaving treatment.

Hence there is a need for a monitoring device and a method that can identify the risk factors early during the initial crucial period, allowing enough time to institute the appropriate care. This method and device should address all of the identified causes of maternal death, should be simple, portable, and cheap and should have the capability to be used in any place regardless of limited resources.

### SUMMARY

This disclosure relates generally to a maternity monitoring device and method. One implementation of the teachings herein is an apparatus for monitoring a plurality of parameters of a user that includes a first sensor adapted to be applied to a chest wall below an axilla of the user; a cord including a first end and a second end, the first end to the first sensor; a second sensor connected to the second end of the cord, the second sensor adapted to be placed over an inner aspect of the upper arm against a major artery of the user; an inflatable cuff adapted to be placed around the upper arm of the user and secure the second sensor against the major artery of the user; and a digital screen disposed on the inflatable cuff, the display adapted to display at least one of the plurality of parameters monitored by the apparatus.

One implementation of the teachings herein is a method for monitoring a plurality of parameters of a user that includes attaching a first sensor of a monitoring device to a chest wall below an axilla of the user; attaching a second sensor disposed on an inflatable cuff of the monitoring device over a major artery of an upper arm of the user; securing the inflatable cuff around the upper arm of the user; receiving data signals from the first sensor and the second sensor of the monitoring device, the data signals comprising at least one of the plurality of parameters; and displaying the at least one of the plurality of parameters, a date, and a time on a digital screen of the monitoring device.

These and other aspects of the present disclosure are disclosed in the following detailed description of the embodiments, the appended claims and the accompanying figures.

### BRIEF DESCRIPTION OF THE DRAWINGS

The various features, advantages, and other uses of the device and method will become more apparent by referring to the following detailed description and drawings, wherein like reference numerals refer to like parts throughout the several views. It is emphasized that, according to common practice, the various features of the drawings are not to-scale. On the contrary, the dimensions of the various features are arbitrarily expanded or reduced for clarity.

FIG. 1 is a perspective view of a first embodiment of a monitoring device, shown worn by a user in accordance with implementations of this disclosure;

FIG. 2 is a elevation view of the first embodiment of the monitoring device, showing a cuff in an unwrapped position;

FIG. 3 detail front elevation view of a display of the first embodiment of the monitoring device, showing temperature data of the user, in accordance with implementations of this disclosure;

FIG. 4 is a detail front elevation view of the display of the first embodiment of the monitoring device, showing respiration data of the user, in accordance with implementations of this disclosure;

FIG. 5 is a detail front elevation view of the display of the first embodiment of the monitoring device, showing pulse data of the user, in accordance with implementations of this disclosure;

FIG. 6 is a detail front elevation view of the display of the first embodiment of the monitoring device, showing blood pressure data of the user, in accordance with implementations of this disclosure; and

FIG. 7 is a flow diagram of a process for monitoring a plurality of physical signs of a user in accordance with implementations of this disclosure.

### DETAILED DESCRIPTION

Every day approximately 830 women die from preventable causes related to pregnancy and childbirth. About 99% of all maternal deaths occur in developing countries. Maternal mortality is higher in women living in rural areas and among poor communities due to the lack of available resources. There are many factors that contribute to maternal death, such as failure to recognize the seriousness of a patient's condition, lack of knowledge, failure to follow recommended practices, lack of or failure to implement policies, protocols and guidelines, and poor organizational arrangements in low resource settings, and diagnostic errors, delay in diagnosis, inappropriate or inadequate treatment, and an inability to get the skilled obstetrician at the patient's bedside during acute emergencies in developed areas. Breakdowns in communication and failures of organizational culture and teamwork have consistently ranked among the top three leading contributors to reported maternal and newborn sentinel events.

There is a need for a monitoring device and a method that can identify the risk factors early during the initial crucial period, allowing enough time to institute the appropriate care. This transition period is the crucial time of intervention in order to prevent maternal death. The monitoring device and method of the present disclosure can identify the important changes in the vital signs of laboring patients and signals abnormal values, trends, and patterns to alert the care provider to intervene. The monitoring device is simple, portable, and low cost, and has the capability to be used in any location regardless of limited resources.

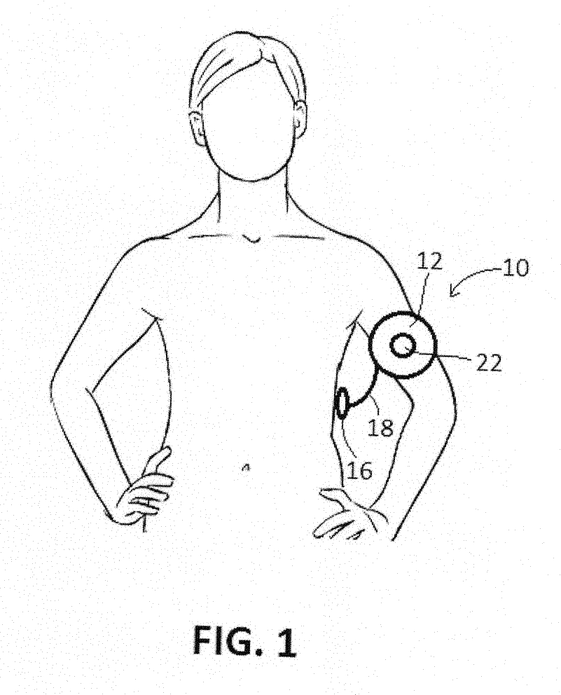

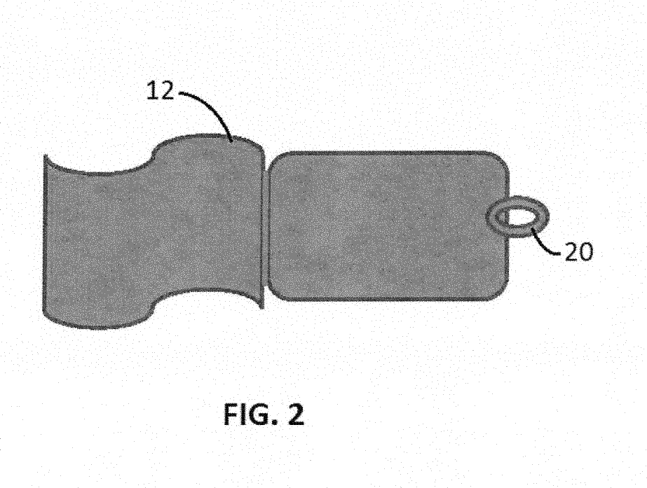

Referring to FIG. 1, a first embodiment of a monitoring device 10 is shown on a patient. The monitoring device 10 comprises an inflatable cuff 12 (FIGS. 1 and 2) and two sensors 14 (not shown), 16 (FIG. 1). Sensor 14 is disposed on the inflatable cuff 12 such that when the cuff 12 is wrapped around the upper arm of the patient, sensor 14 is placed over the inner aspect of the upper arm against a major artery, such as the brachial artery. Sensor 14, when the cuff 12 is inflated and deflated, can check the patient's blood pressure and pulse. The cuff 12 comes in various sizes to accommodate the patient's arm size and includes hook and look fasteners (not shown) allowing the cuff 12 to be wrapped and secured around the arm of the patient. Sensor 16 is applied to the chest wall of the patient, just below the axilla, and is connected to sensor 14 via a loose cord 18 that will not limit the patient's arm movement. Sensor 16 is configured to check the patient's temperature and count the patient's chest wall movements with respiration, thereby monitoring the patient's respiratory rate.

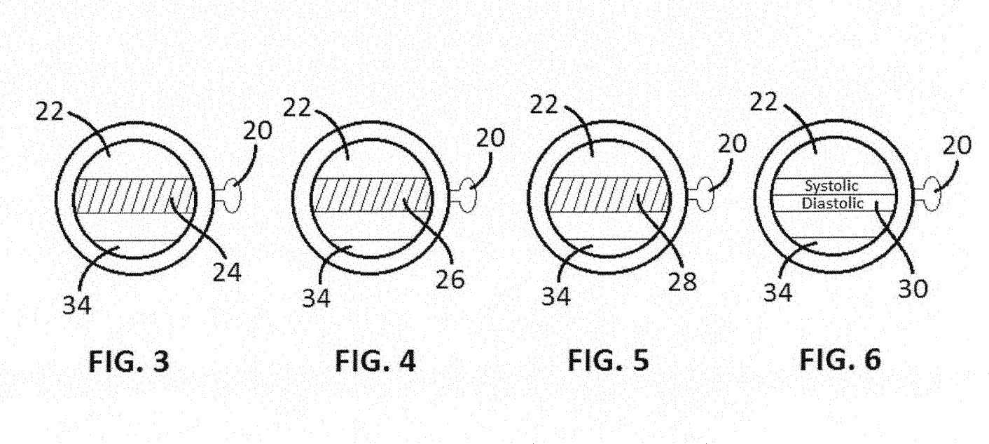

In this implementation, the monitoring device 10 can also comprise a dial 20, shown in FIGS. 2-6, that can be turned to display a plurality of parameters on a display 22, shown in FIGS. 1 and 3-6. The rotatable dial 20 allows the user to select between parameters and additional programming options. The plurality of parameters in this exemplary implementation includes pulse, temperature, respiratory rate, and blood pressure. In this implementation, the dial 20 has four settings that each display a vital sign parameter of the plurality of parameters in a graphic or numerical value format and a date and time display 34, shown in FIGS. 3-6, on the display 22 and one additional setting that allows for additional device programming. A first setting, as shown in FIG. 3, displays the patient's pulse in beats per minute and can also be displayed in graph form on the display 22, allowing a medical professional to easily identify an upward or downward trend when displayed as a graph as time over value. If the patient's pulse is within normal range, the pulse will display in the normal pulse range 24 of display 22. A second setting, as shown in FIG. 4, displays the patient's temperature in Centigrade or Fahrenheit and can also be displayed in graph form on the display 22, allowing the medical professional to easily identify an upward or downward trend when displayed as a graph as time over value. If the patient's temperature is within normal range, the temperature will display in the normal temperature range 26 of display 22. A third setting, as shown in FIG. 5, displays the patient's respiratory rate per minute and can also be displayed in graph form on the display 22, allowing the medical professional to easily identify an upward or downward trend when displayed as a graph as time over value. If the patient's respiratory rate is within normal range, the respiratory rate will display in the normal respiratory rate range 28 of display 22. A fourth setting, as shown in FIG. 6, displays the patient's blood pressure as systolic pressure over diastolic pressure on the display 22, measured in millimeters of mercury (mmHg), above the surrounding atmospheric pressure (considered to be zero for convenience). The systolic and diastolic pressures can also be displayed in graph form on the display 22, allowing the medical professional to easily identify an upward or downward trend when displayed as a graph as time over value. If the patient's blood pressure is within normal range, the blood pressure will display in the normal blood pressure range 30 of display 22.

The monitoring device 10 includes a built-in battery and a software application that can be programmed to check various vital signs, or plurality of parameters, at predetermined or customized time intervals or as often as needed. The monitoring device 10 also comprises a built-in automatic alert system for detecting abnormal values based on given parameters. Alert parameters for each of the vital sign plurality of parameters, for the first setting 24, the second setting 26, the third setting 28, and the fourth setting 30, will be set at upper and lower normal ranges. Any value that is above an upper normal range or below a lower normal range will automatically be rechecked in a configurable or predetermined amount of time, such as ten minutes. Once rechecked, the monitoring device 10 will set off an alarm, such as a beep, if any parameter remains outside of the normal range. Any of the patient's vital sign parameters (pulse, temperature, blood pressure, respiratory rate, etc.) can be checked at any interval and the monitoring device 10 can be programmed to monitor additional user parameters using the additional device programming setting 32 (not shown) on dial 20.

The software application can also include an automatic messaging system that provides the patient's parameters and/or history to any designated center via a variety of wireless communications links. The wireless data link can be, for example, a wireless local area network (WLAN), wireless metropolitan area network (WMAN), wireless wide area network (WWAN), a private wireless system, a cellular telephone network or any other means of transferring data and/or messages from the monitoring device 10 to, in this example, a remote designated center. The monitoring device 10 can also connect via wireless access to a smart electronic obstetrical health record system and transmit and/or store the patient's parameters and/or history.

The monitoring device 10 can be used as a stand-alone system with the built-in alert/alarm system and automatic messaging system. The monitoring device 10 can also be used with a smartphone and/or a computer, where the device 10 can instantly connect to a cloud-based software system and generate instant pop-ups with diagnoses and up-to-date and detailed management guidelines. The cloud-based software system can be uploaded to or integrated with the monitoring device 10, which helps the provider in patient management.

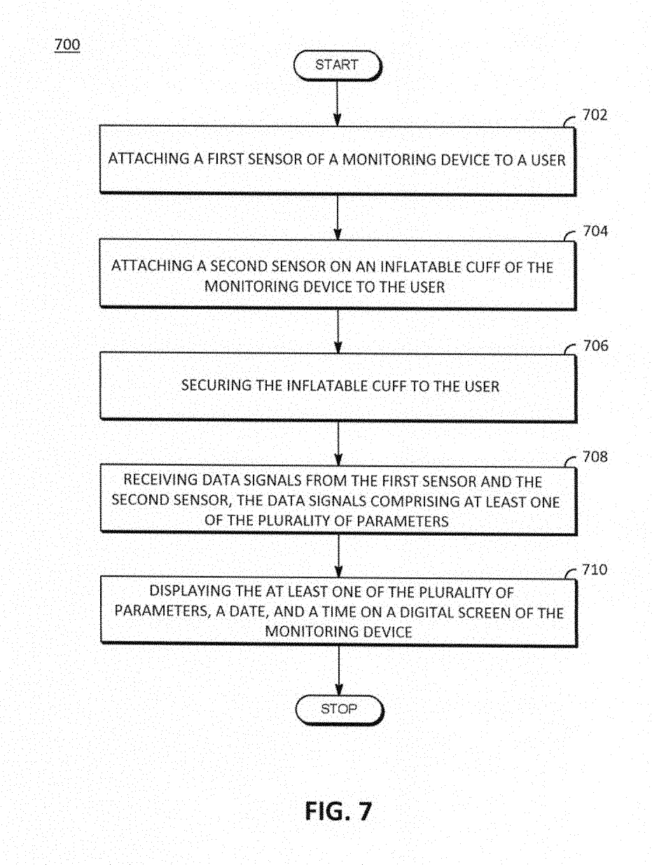

In use, the person initiating the monitoring device 10 keys in the patient's name, date of birth, and thumb print to create a unique ID for that patient. FIG. 7 is a flow diagram showing a process 700 for monitoring a plurality of parameters of a user in accordance with an implementation of this disclosure. The monitoring device 10 is placed on the patient by attaching the sensor 16 of a monitoring device 10 to the chest wall below an axilla of the patient 702. Sensor 14 is then placed over the inner aspect of the upper arm of the patient over a major artery of the patient 704. The inflatable cuff 12 is secured around the upper arm of the patient 706. The monitoring device 10 receives data signals from the sensor 16 and the sensor 14, where the data signals comprises at least one of the plurality of parameters 708. The monitoring device 10 then displays the at least one of the plurality of parameters, a date, and a time 710 on the display 22. The monitoring device 10 then automatically records and stores the patient's vital signs within monitoring device 10, sends alerts when values are abnormal, and sends automatic messages. The monitoring device 10 also provides pop-ups with up-to-date treatment guidelines when connected through a computer or mobile device to the electronic obstetrical health record system through the wireless connection. The data recorded and stored within the monitoring device 10 is time sensitive, accurate, safe, and secure, complying with the requirements of the Health Insurance Portability and Accountability Act of 1996 (HIPPA). The monitoring device 10 can send instant messages to a designated center with emergency care facilities, which can be crucial in cases where the patient is located in a low resources center or location.

While the present disclosure has been described in connection with certain embodiments, it is to be understood that the invention is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

1. An apparatus for monitoring a plurality of parameters of a user comprising:
a first sensor adapted to be applied to a chest wall below an axilla of the user;
a cord including a first end and a second end, the first end to the first sensor;
a second sensor connected to the second end of the cord, the second sensor adapted to be placed over an inner aspect of the upper arm against a major artery of the user;
an inflatable cuff adapted to be placed around the upper arm of the user and secure the second sensor against the major artery of the user; and
a digital screen disposed on the inflatable cuff, the display adapted to display at least one of the plurality of parameters monitored by the apparatus.

2. The apparatus of claim 1, further comprising:
hook fastening material and loop fastening material disposed on opposite sides of the inflatable cuff, the hook fastening material and the loop fastening material adapted to removably fasten the inflatable cuff around the upper arm of the user.

3. The apparatus of claim 1, further comprising:
a rotatable dial in electronic communication with the digital screen, the dial adapted to select one of the plurality of parameters to display on the digital screen.

4. The apparatus of claim 3, wherein the rotatable dial is adapted to select additional programmable settings for the apparatus.

5. The apparatus of claim 4, wherein the additional programmable settings are configurable to monitor additional user data.

6. The apparatus of claim 1, wherein the digital screen is adapted to display a date and a time.

7. The apparatus of claim 1, wherein the first sensor is adapted to monitor at least one of a temperature and a respiratory rate of the user and the second sensor is adapted to monitor at least one of a pulse and a blood pressure of the user.

8. The apparatus of claim 1, wherein the digital screen is adapted to display one of the plurality of parameters within a normal range location on the digital screen on a condition that the one of the plurality of parameters is within normal range.

9. The apparatus of claim 1, wherein the digital screen is adapted to display one of the plurality of parameters in one of a numerical format and a graphical format, the graphical format representing time over value.

10. The apparatus of claim 1, further comprising:
a software application adapted to monitor the plurality of parameters using data from the first sensor and the second sensor at predetermined time intervals, wherein the time intervals are configurable.

11. The apparatus of claim 1, further comprising:
an automatic alert system adapted to set off an alarm on a condition that at least one of the plurality of parameters are outside a normal range for that parameter for a predetermined amount of time.

12. The apparatus of claim 1, further comprising:
an automatic messaging system adapted to send at least one of the plurality of parameters to a designated center over a wireless data link.

13. The apparatus of claim 1, further comprising:
a wireless communications link adapted to communicate with at least one of a computer and a mobile device to access a cloud-based obstetric software system, wherein the cloud-based obstetric software system is adapted to generate pop-up messages based on an alert received from the monitoring device, the pop-up messages comprising at least one of diagnoses and management guidelines.

14. The apparatus of claim 13, wherein the cloud-based obstetric software system is adapted to provide two-way communication between a designated center and a remote center on a condition that the cloud-based obstetric software system is wirelessly connected to the designated center.

15. A method for monitoring a plurality of parameters of a user comprising:
attaching a first sensor of a monitoring device to a chest wall below an axilla of the user;
attaching a second sensor of the monitoring device over a major artery of an upper arm of the user;
securing an inflatable cuff around the upper arm of the user;
receiving data signals from the first sensor and the second sensor of the monitoring device, the data signals comprising at least one of the plurality of parameters; and
displaying the at least one of the plurality of parameters, a date, and a time on a digital screen of the monitoring device.

16. The method of claim 15, further comprising:
selecting one of a plurality of settings on a dial of the monitoring device; and
displaying one of the plurality of parameters, date, and time based on the setting selected on the dial.

17. The method of claim 16, wherein the plurality of settings comprises one of pulse, temperature, respiratory rate, blood pressure, and additional programmable settings.

18. The method of claim 15, further comprising:
monitoring, using the first sensor of the monitoring device, at least one of a temperature and a respiratory rate of the user; and
monitoring, using the second sensor of the monitoring device, at least one of a pulse and a blood pressure of the user.

19. The method of claim 15, wherein displaying at least one of the plurality of parameters, date, and time on the digital screen comprises displaying the at least one of the plurality of parameters, date, and time in one of a numerical format and a graphical format, the graphical format representing time over value.

20. The method of claim 15, further comprising:
monitoring the plurality of parameters, using a software application of the monitoring device, at predetermined time intervals, wherein the predetermined time intervals are configurable.

21. The method of claim 15, further comprising:
sending messages, using a cloud-based software system interacting with the monitoring device, to at least one of a computer and a mobile device; and
providing two-way communication, using the cloud-based software system, between the monitoring device and at least one of the computer and the mobile device at a designated center.

22. The method of claim 15, further comprising:
sending, using an automatic messaging system on the monitoring device, at least one of the plurality of parameters to a remote center over a wireless data link.

23. The method of claim 15, further comprising:
activating an alarm, using an automatic alert system on the monitoring device, on a condition that at least one of the plurality of parameters is outside a normal range for that parameter for a predetermined amount of time.

24. The method of claim 15, further comprising:
securing the inflatable cuff, using a hook fastening material and a loop fastening material on opposite sides on the inflatable cuff, to the upper arm of the user.

25. The method of claim 15, wherein displaying at least one of the plurality of parameters on the digital screen comprises displaying the at least one of the plurality of parameter within a normal range location on the digital screen on a condition that the parameter is within normal range.
